SEQUENCE LISTING (9 sequences)

SEQ ID NO: 1 - protein, 610 aa, Homo sapiens
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 2 - DNA, 3875 bp, Homo sapiens
  source 1..3875: mol_type = other DNA
agctgttcttggctgacttcacatcaaaactcctatactgacctgagacagaggcagcagtgatacccacctgagagatcctgtgtttgaacaactgcttcccaaaacggaaagtatttcaagcctaaacctttgggtgaaaagaactcttgaagtcatgattgcttcacagtttctctcagctctcactttggtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctccaggtgaacccaacaataggcaaaaagatgaggactgcgtggagatctacatcaagagagaaaaagatgtgggcatgtggaatgatgagaggtgcagcaagaagaagcttgccctatgctacacagctgcctgtaccaatacatcctgcagtggccacggtgaatgtgtagagaccatcaataattacacttgcaagtgtgaccctggcttcagtggactcaagtgtgagcaaattgtgaactgtacagccctggaatcccctgagcatggaagcctggtttgcagtcacccactgggaaacttcagctacaattcttcctgctctatcagctgtgataggggttacctgccaagcagcatggagaccatgcagtgtatgtcctctggagaatggagtgctcctattccagcctgcaatgtggttgagtgtgatgctgtgacaaatccagccaatgggttcgtggaatgtttccaaaaccctggaagcttcccatggaacacaacctgtacatttgactgtgaagaaggatttgaactaatgggagcccagagccttcagtgtacctcatctgggaattgggacaacgagaagccaacgtgtaaagctgtgacatgcagggccgtccgccagcctcagaatggctctgtgaggtgcagccattcccctgctggagagttcaccttcaaatcatcctgcaacttcacctgtgaggaaggcttcatgttgcagggaccagcccaggttgaatgcaccactcaagggcagtggacacagcaaatcccagtttgtgaagctttccagtgcacagccttgtccaaccccgagcgaggctacatgaattgtcttcctagtgcttctggcagtttccgttatgggtccagctgtgagttctcctgtgagcagggttttgtgttgaagggatccaaaaggctccaatgtggccccacaggggagtgggacaacgagaagcccacatgtgaagctgtgagatgcgatgctgtccaccagcccccgaagggtttggtgaggtgtgctcattcccctattggagaattcacctacaagtcctcttgtgccttcagctgtgaggagggatttgaattacatggatcaactcaacttgagtgcacatctcagggacaatggacagaagaggttccttcctgccaagtggtaaaatgttcaagcctggcagttccgggaaagatcaacatgagctgcagtggggagcccgtgtttggcactgtgtgcaagttcgcctgtcctgaaggatggacgctcaatggctctgcagctcggacatgtggagccacaggacactggtctggcctgctacctacctgtgaagctcccactgagtccaacattcccttggtagctggactttctgctgctggactctccctcctgacattagcaccatttctcctctggcttcggaaatgcttacggaaagcaaagaaatttgttcctgccagcagctgccaaagccttgaatcagatggaagctaccaaaagccttcttacatcctttaagttcaaaagaatcagaaacaggtgcatctggggaactagagggatacactgaagttaacagagacagataactctcctcgggtctctggcccttcttgcctactatgccagatgcctttatggctgaaaccgcaacacccatcaccacttcaatagatcaaagtccagcaggcaaggacggccttcaactgaaaagactcagtgttccctttcctactctcaggatcaagaaagtgttggctaatgaagggaaaggatattttcttccaagcaaaggtgaagagaccaagactctgaaatctcagaattccttttctaactctcccttgctcgctgtaaaatcttggcacagaaacacaatattttgtggctttctttcttttgcccttcacagtgtttcgacagctgattacacagttgctgtcataagaatgaataataattatccagagtttagaggaaaaaaatgactaaaaatattataacttaaaaaaatgacagatgttgaatgcccacaggcaaatgcatggagggttgttaatggtgcaaatcctactgaatgctctgtgcgagggttactatgcacaatttaatcactttcatccctatgggattcagtgcttcttaaagagttcttaaggattgtgatatttttacttgcattgaatatattataatcttccatacttcttcattcaatacaagtgtggtagggacttaaaaaacttgtaaatgctgtcaactatgatatggtaaaagttacttattctagattaccccctcattgtttattaacaaattatgttacatctgttttaaatttatttcaaaaagggaaactattgtcccctagcaaggcatgatgttaaccagaataaagttctgagtgtttttactacagttgttttttgaaaacatggtagaattggagagtaaaaactgaatggaaggtttgtatattgtcagatattttttcagaaatatgtggtttccacgatgaaaaacttccatgaggccaaacgttttgaactaataaaagcataaatgcaaacacacaaaggtataattttatgaatgtctttgttggaaaagaatacagaaagatggatgtgctttgcattcctacaaagatgtttgtcagatatgatatgtaaacataattcttgtatattatggaagattttaaattcacaatagaaactcaccatgtaaaagagtcatctggtagatttttaacgaatgaagatgtctaatagttattccctatttgttttcttctgtatgttagggtgctctggaagagaggaatgcctgtgtgagcaagcatttatgtttatttataagcagatttaacaattccaaaggaatctccagttttcagttgatcactggcaatgaaaaattctcagtcagtaattgccaaagctgctctagccttgaggagtgtgagaatcaaaactctcctacacttccattaacttagcatgtgttgaaaaaaaagtttcagagaagttctggctgaacactggcaacaacaaagccaacagtcaaaacagagatgtgataaggatcagaacagcagaggttcttttaaaggggcagaaaaactctgggaaataagagagaacaactactgtgatcaggctatgtatggaatacagtgttattttctttgaaattgtttaagtgttgtaaatatttatgtaaactgcattagaaattagctgtgtgaaataccagtgtggtttgtgtttgagttttattgagaattttaaattataacttaaaatattttataatttttaaagtatatatttatttaagcttatgtcagacctatttgacataacactataaaggttgacaataaatgtgcttatgttta

SEQ ID NO: 3 - protein, 619 aa, Mus musculus
MKATAGVMNASRFLSALVFVLLAGESTAWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRPLVVALSAAGTSLLTSSSLLYLLMRYFRKKAKKFVPASSCQSLQSFENYHVPSYNV

SEQ ID NO: 4 - protein, 549 aa, Rattus norvegicus
MNASCFLSALTFVLLIGKSIAWYYNASSELMTYDEASAYCQRDYTHLVAIQNKEEINYLNSTLRYSPSYYWIGIRKVNNVWIWVGTQKPLTEEAKNWAPGEPNNKQRNEDCVEIYIQRPKDSGMWNDERCDKKKLALCYTASCTNTSCSGHGECVETINSYTCKCHPGFLGPKCDQVVTCQEQEYPDHGSLNCTHPFGLFSYNSSCSFSCERGYVPSSMETTVRCTSSGEWSAPAPACHVVECKALTQPAHGVRKCSSNPGSYPWNTTCTFDCEEGYRRVGAQNLQCTSSGVWDNEKPSCKAVTCDAIPRPQNGSVSCSNSTAGALAFKSSCNFTCEHSFTLQGPAQVECSAQGQWTPQIPVCKASQCEALSAPQRGHMKCLPSASAPFQSGSSCKFSCDEGFELKGSRRLQCGPRGEWDSEKPTCAGVQCSSLDLPGKMNMSCSGPAVFGTVCEFTCPEGWTLNGSSILTCGATGRWSAMLPTCEAPANPPRPLVVALSVAATSLLTLSSLIYVLKRFFWKKAKKFVPASSCQSLQSFENYQGPSYII

SEQ ID NO: 5 - protein, 611 aa, Canis lupus
MITSQLLPALTLVLLLFKEGGAWSYNASTEAMTFDEASTYCQQRYTHLVAIQNQEEIKYLNSMFTYTPTYYWIGIRKVNKKWTWIGTQKLLTEEAKNWAPGEPNNKQNDEDCVEIYIKRDKDSGKWNDERCDKKKLALCYTAACTPTSCSGHGECVETVNNYTCKCHPGFRGLRCEQVVTCQAQEAPEHGSLVCTHPLGTFSYNSSCFVSCDKGYLPSSTEATQCTSTGEWSASPPACNVVECSALTNPCHGVMDCLQSSGNFPWNMTCTFECEEGFELMGPKRLQCTSSGNWDNRKPTCKAVTCGAIGHPQNGSVSCSHSPAGEFSVRSSCNFTCNEGFLMQGPAQIECTAQGQWSQQVPVCKASQCKALSSPERGYMSCLPGASGSFQSGSSCEFFCEKGFVLKGSKTLQCGLTGKWDSEEPTCEAVKCDAVQQPQDGLVRCAHSSTGEFTYKSSCAFSCEEGFELHGSAQLECTSQGQGVTGGPSCQVVQCFKSGSFRKDEHKLQGEPVFGAVCAFACPEGWTLNGSAALMCDATGHWSGMLPTCEAPTESSIPLAVGLTAGGTSLLTVASFLLWLLKRLRKRAKKFVPASSCQSLQSDGSYHMPCSI

SEQ ID NO: 6 - protein, 484 aa, Ovis aries
MIASQYLSALTFVLLFKESRTWSYHASTEIMTFEAARDYCQKTYTALVAIQNQEEIEYLNSTFSHSPSYYWIGIRKINGTWTWIGTNKSLTEEATNWAPGEPNNKQTDEDCVEIYIKREKDSGKWNDENCTKKKFALCYKAACTPTSCSSHGECVETINSHTCQCHPGFKGLRCEQVVTCQAQKHPEHGHLVCNPLGKFTYNSSCSISCAEGYLPSSTEAARCMSSGEWSTPLPKCNVVKCDALSNPDNGVVNCSQNHGSLPWNTTCMFECQEGYKLTGPQHLQCTSSGIWDNKQPTCKAVTCAAISHPQNGTVKCSHSLVGDFAFKSSCHFTCAEGFALQGPAQVECTAQGRWTQQVPVCEVVRCSRLDVSGKLNMDCNGEPVLGTECMFACPEGWTLNGSVALTCGATGHWSGMLPTCEAPTVSQTPLAVGLSTAGVSLVTIPSFLFWLLKRLQMKAEKLSDDSSCSSLKSNGCYSTPSELV

SEQ ID NO: 7 - protein, 21 aa, Homo sapiens
MIASQFLSALTLVLLIKESGA

SEQ ID NO: 8 - protein, 589 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 9 - DNA, 1860 bp, Mus musculus
  source 1..1860: mol_type = other DNA
atgaaagcaactgctggagtcatgaatgcctcgcgctttctctctgctcttgtttttgttctcctcgctggagagagcacagcttggtactacaatgcctccagtgagctcatgacgtatgatgaagccagtgcatactgtcagcgggactacacacatctggtggcgattcagaacaaggaagagatcaactaccttaactccaatctgaaacattcaccgagttactactggattggaatcagaaaagtcaataacgtatggatctgggtggggacggggaagcctctgacagaggaagctcagaactgggctccaggtgaaccaaacaacaaacaaagaaatgaggactgtgtagagatttacatccaacgaaccaaagactcgggcatgtggaatgacgagagatgtaacaaaaagaagctggctctgtgctacacagcttcgtgtaccaatgcatcctgcagtggtcatggtgaatgcatagagaccatcaatagttacacctgcaagtgccaccctggcttcctgggacccaactgtgagcaagctgtgacttgcaaaccacaggaacaccctgactatggaagcctgaactgctcccacccgttcggccccttcagctataattcctcctgctcctttggctgtaaaaggggctacctgcccagcagcatggagaccaccgtgcggtgtacgtcctctggagagtggagtgcgcctgctccagcctgccatgtggttgaatgtgaagctttgacccaccctgcccacggtatcaggaaatgttcctcaaatcctgggagctacccatggaacacgacatgcacgtttgactgtgtggaagggtacaggcgagttggagctcagaatctacagtgtacctcatctggcatctgggataacgagacgccatcatgcaaagctgtgacctgtgacgccatccctcagcctcagaatggctttgtgagctgcagccactcaacagctggagaacttgcgtttaagtcatcctgtaacttcacctgtgagcagagtttcacgttgcaggggccagcgcaggttgaatgcagcgcacaagggcagtggacaccacaaatcccagtctgcaaagctgtccagtgtgaagccttatctgcgccacagcagggcaacatgaaatgtcttcccagtgcttctggacctttccaaaatgggtccagttgtgagttctcctgcgaagaaggatttgaactgaagggatcaagaagacttcagtgtggtccaagaggggaatgggatagcaagaagcccacgtgttcagctgtgaaatgtgatgatgtccctcggccccagaatggcgtcatggagtgtgctcatgctactactggagaattcacctacaagtcctcatgtgcctttcaatgcaatgagggctttagcttgcatggctcagctcaacttgagtgcacatctcagggaaagtggacccaggaagtcccctcctgccaagtggtacaatgtccaagccttgacgtcccgggaaagatgaacatgagctgcagcggaacagcagttttcggcacagtgtgtgagtttacatgtcctgatgattggacactcaatggatctgcagttctgacgtgtggtgccacgggacgctggtctgggatgccgcctacctgtgaagccccagtcagccccacccgtcccttggtagttgcactttctgcggcaggaacctcactcctgacatcgtcctcattgctctacttgttgatgagatactttcggaagaaagcaaagaaatttgttcctgctagcagctgccaaagccttcaatcatttgaaaactaccatgtgccttcttacaacgtctag

(19) United States
(12) Patent Application Publication
(10) Pub. No.: US 2026/0256946 A1
(43) Pub. Date: Sep. 3, 2026

(54) METHOD FOR PROMOTING MYOGENESIS AND SKELETAL MUSCLE RECOVERY

(71) Applicant: UNIVERSITY OF MIAMI, Miami, FL (US)

(72) Inventors: Zhao-Jun Liu, Miami, FL (US); Omaida Velazquez, Miami, FL (US)

(21) Appl. No.: 19/474,562

(22) PCT Filed: Apr. 12, 2024
(86) PCT No.: PCT/US24/24434

§ 371 Date: Oct. 10, 2025

Related U.S. Application Data
(60) Provisional application No. 63/459,192, filed on Apr. 13, 2023.
(60) Provisional application No. 63/465,015, filed on May 9, 2023.

(51) Int. Cl. A61K 48/00; A61K 38/17; A61P 21/00; C12N 15/86

(52) U.S. Cl. CPC A61K 48/005; A61K 38/178; A61K 48/0075; A61P 21/00; C12N 15/86; C12N 2750/14143

(57) ABSTRACT

The disclosure relates to, in part, to methods of inducing myogenesis in non-ischemic atrophied muscle in a subject in need thereof, comprising administering to the subject a hybrid adeno-associated virus (AAV) comprising a nucleotide sequence encoding an E-selectin, in amount effective to induce myogenesis in the non-ischemic atrophied muscle of the subject.

### GOVERNMENT SUPPORT CLAUSE

This invention was made with government support under Grant No. HL156152 and HHSN268201700008C awarded by the National Institutes of Health. The Government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 58947_SeqListing.xml; Size: 17,725 bytes; Created Apr. 12, 2024), which is incorporated by reference in its entirety.

### BACKGROUND

Skeletal muscle atrophy or muscle wasting are a nearly universal consequence of severe human illnesses, including cancer, chronic renal failure, congestive heart failure, chronic respiratory disease, insulin deficiency, acute critical illness, chronic infections such as HIV/AIDS, muscle denervation, medications such as Glucagon-like Peptide 1 (GLP-1) Agonists, and many other medical and surgical conditions that limit muscle use.

Despite advances in understanding the physiology and pathophysiology of muscle atrophy or muscle wasting, there is still a scarcity of therapies that are effective in the treatment, reversal or slowing of muscle atrophy or wasting associated with diseases in which the muscle atrophies or the need to increase muscle mass is involved.

### SUMMARY

The present disclosure provides methods and compositions for inducing myogenesis in non-ischemic atrophied muscle in a subject in need thereof, comprising administering to the subject a hybrid adeno-associated virus (AAV) comprising a nucleotide sequence encoding an E-selectin, in amount effective to induce myogenesis in the non-ischemic atrophied muscle of the subject.

In another aspect, the disclosure provides methods and compositions for treating a muscle tear in non-ischemic muscle tissue in a subject in need thereof, comprising administering to the subject a hybrid adeno-associated virus (AAV) comprising a nucleotide sequence encoding an E-selectin, in amount effective to treat the muscle tear in the subject.

In another aspect, the disclosure provides methods and compositions for improving the outcome of a connective tissue reattachment procedure in a subject in need thereof comprising administering to the subject a hybrid adeno-associated virus (AAV) comprising a nucleotide sequence encoding an E-selectin in an amount effective to improve the outcome of procedure.

In aspects, the present disclosure also provides methods and compositions for directing AAV2 to deliver E-selectin to skeletal muscle to improve perfusion and exercise performance.

In aspects, the present disclosure provides E-selectin/AAV gene therapy that promotes myogenesis and skeletal muscle recovery in, for instance, mouse hindlimb ischemia model.

In another aspect, the present disclosure provides E-selectin/AAV gene therapy for improving myogenesis (muscle regeneration), decreased atrophy, and functional recovery of skeletal muscle in ischemic limbs.

In yet another aspect, the present disclosure provides E-selectin/AAV gene therapy as a nonsurgical adjunct in patients with life-limiting PAD.

The details of the disclosure are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

As used herein, all headings are simply for organization and are not intended to limit the disclosure in any manner. The content of any individual section may be equally applicable to all sections.

### BRIEF DESCRIPTION OF THE DRAWINGS

FIGS. 1A-1B show a non-limiting schematic representation of intraoperative images of the murine hindlimb vasculature. Coagulation of the femoral artery (FA) and vein (FV) is performed (FIG. 1A) proximally and (FIG. 1B) distally with preservation of the femoral nerve (FN) as indicated by the forceps. PCFA, proximal caudal femoral artery; LCFA, lateral circumflex femoral artery; PA, popliteal artery; SA, saphenous artery; SCEA, superficial caudal epigastric artery.

FIGS. 2A-2C show (FIG. 2A) Grip strength meter with (FIGS. 2B and 2C) positioning of mouse on grid assembly.

FIGS. 3A-3B show a non-limiting schematic representation of E-sel/AAV induces high-level transgene expression. (FIG. 3A) Immunofluorescence pattern of E-selectin (CD62E) expression in ischemic muscle with some CD62E⁺ cells forming capillaries (arrows). (FIG. 3B) E-sel mRNA levels in ischemic muscle are 322-fold higher three weeks after treatment with E-sel/AAV compared to LacZ/AAV (N=4 per group). Data are presented as mean±SEM where ***P<0.001.

FIGS. 4A-4B show a non-limiting schematic representation of E-sel/AAV enhances reperfusion of ischemic muscle. (FIG. 4A) Representative laser Doppler perfusion images with (FIG. 4B) quantification of perfusion indices demonstrating improved recovery of footpad perfusion in mice treated with E-sel/AAV compared to LacZ/AAV (N=20 per group). Data are presented as mean±SEM where **P<0.01 and ***P<0.001.

FIGS. 5A-5C show a non-limiting schematic representation of E-sel/AAV enhances functional recovery of ischemic muscle. (FIG. 5A) Mean and (FIG. 5B) peak postoperative hindlimb grip strengths are increased in mice treated with E-sel/AAV compared to LacZ/AAV (N=20 per group). (FIG. 5C) Maximal distance travelled on treadmill exhaustion testing is increased in mice treated with E-sel/AAV compared to LacZ/AAV (N=8 per group). Data are presented as mean±SEM where *P<0.05 and **P<0.01.

FIGS. 6A-6B show a non-limiting schematic representation of E-sel/AAV enhances proliferation of myogenic precursors in ischemic muscle. (FIG. 6A) Representative immunofluorescence images and (FIG. 6B) quantification of MyoD and Ki-67 expression demonstrating increased number of MyoD⁺/Ki-67+ myogenic precursors (white arrow) in ischemic gastrocnemius muscle treated with E-sel/AAV compared to LacZ/AAV (N=5 per group). In comparison, few MyoD⁺ or Ki-67⁺ cells are identified in non-treated, non-ischemic muscle from either group. Scale bars represent 50 μm. Data are presented as mean±SEM where *P<0.05 and ***P<0.001.

FIGS. 7A-7B show a non-limiting schematic representation of E-sel/AAV is associated with increased Myh7⁺ myofibers in regenerated skeletal muscle. (FIG. 7A) Representative immunofluorescence images and (FIG. 7B) quantification demonstrating increased proportion of Myh7⁺ myofibers in ischemic gastrocnemius muscle treated with E-sel/AAV compared to LacZ/AAV (N=5 per group). Data are presented as mean±SEM where ***P<0.001.

FIGS. 8A-8B show that overexpression of E-Selectin on the cell-membrane of human Skeletal Muscle Cells activates these cells to increase their proliferation (regenerative capacity) and their energy as measured by their ATP content. Human skeletal muscle cells (hSkMC) were transduced with E-selectin viral vector gene transfer or GFP gene transfer as a control. (FIG. 8A) Following transduction, 5×10⁴ E-selectin+-hSkMC and GFP+-hSkMC were seeded in 6-well plates and proliferation was assessed by cell counting. Transgene expression was confirmed by flow cytometry and ATP content was measured after lysis of 1×10⁶ cells. Cell counting showed enhanced proliferation of E-selectin+-hSkMC (18.3±3.3×10⁴ cells) by Day 7 compared to GFP+-hSkMC controls (11.8±1.5×10⁴ cells) (p=0.02). (FIG. 8B) There was also significantly greater ATP content in E-selectin+-hSkMC (1.48±0.03 nmol) compared to GFP+-hSkMC (1.33±0.04 nmol, p=0.003) and untransduced hSkMC (1.26±0.04 nmol, p<0.001). In vitro overexpression of E-selectin in hSkMC increases both cellular proliferation and ATP content.

FIGS. 9A and 9B show that in vitro virally induced overexpression of E-selectin in human MSC promotes cellular proliferation as measured by microscopy (FIG. 9A) and WST-1 assay (FIG. 9B).

### DETAILED DESCRIPTION

The present disclosure is based, in part, on the finding that E-Selectin/AAV gene therapy promotes myogenesis and skeletal muscle recovery in a mouse hindlimb ischemia model.

In one aspect, described herein is a method of inducing myogenesis in non-ischemic atrophied muscle in a subject in need thereof, comprising administering to the subject a hybrid adeno-associated virus (AAV) comprising a nucleotide sequence encoding an E-selectin, in amount effective to induce myogenesis in the non-ischemic atrophied muscle of the subject.

In some embodiments, the subject is not suffering from peripheral artery disease (PAD). In some embodiments, the subject is not suffering from critical limb ischemia.

### Muscle Atrophy

Muscle atrophy refers to a loss of muscle mass and/or to a progressive weakening and/or degeneration of muscles. In some cases, the loss of muscle mass and/or the progressive weakening and degeneration of muscles occurs due to a high rate of protein degradation, a low rate of protein synthesis, or a combination of both. In some cases, a high rate of muscle protein degradation is due to muscle protein catabolism (i.e., the breakdown of muscle protein in order to use amino acids as substrates for gluconeogenesis). The phrase “non-ischemic atrophied muscle” refers to muscle that has decreased in mass and/or progressive weakening by means other than lack of oxygen.

In some embodiments, muscle atrophy refers to a significant loss in muscle strength. By significant loss in muscle strength is meant a reduction of strength in diseased, injured, or unused muscle tissue in a subject relative to the same muscle tissue in a control subject. In some embodiments, a significant loss in muscle strength is a reduction in strength of at least 10%, at least 15%, at least 20%, at least 25%, at least 30%, at least 35%, at least 40%, at least 45%, at least 50%, or more relative to the same muscle tissue in a control subject. In some embodiments, a significant loss in muscle strength is a reduction of strength in unused muscle tissue relative to the muscle strength of the same muscle tissue in the same subject prior to a period of nonuse. In some embodiments, a significant loss in muscle strength is a reduction of at least 10%, at least 15%, at least 20%, at least 25%, at least 30%, at least 35%, at least 40%, at least 45%, at least 50%, or more relative to the muscle strength of the same muscle tissue in the same subject prior to a period of nonuse.

In some embodiments, muscle atrophy refers to a significant loss in muscle mass. In some embodiments, “significant loss in muscle mass” is a reduction of muscle volume in diseased, injured, or unused muscle tissue in a subject relative to the same muscle tissue in a control subject. In some embodiments, “significant loss of muscle volume” is at least 10%, at least 15%, at least 20%, at least 25%, at least 30%, at least 35%, at least 40%, at least 45%, at least 50%, or more relative to the same muscle tissue in a control subject. In some embodiments, “significant loss in muscle mass” is a reduction of muscle volume in unused muscle tissue relative to the muscle volume of the same muscle tissue in the same subject prior to a period of nonuse. In an embodiment, a significant loss in muscle tissue is at least 10%, at least 15%, at least 20%, at least 25%, at least 30%, at least 35%, at least 40%, at least 45%, at least 50%, or more relative to the muscle volume of the same muscle tissue in the same subject prior to a period of nonuse. Muscle volume is optionally measured by evaluating the cross-section area of a muscle such as by Magnetic Resonance Imaging (e.g., by a muscle volume/cross-section area (CSA) MRI method).

In some embodiments, muscle atrophy is skeletal muscle loss or weakness caused by malnutrition, aging, muscle disuse (such as voluntary and involuntary bed rest), neurologic disease (such as multiple sclerosis, amyotrophic lateral sclerosis, spinal muscular atrophy, critical illness neuropathy, spinal cord injury, peripheral neuropathy, or peripheral nerve injury), injury to the limbs or joints, casting, other post-surgical forms of limb immobilization, or spaceflight), chronic disease (such as cancer, congestive heart failure, chronic pulmonary disease, chronic renal failure, chronic liver disease, diabetes mellitus, glucocorticoid excess, growth hormone deficiency, IGF-I deficiency, estrogen deficiency, and chronic infections such as HIV/AIDS or tuberculosis), burn injuries, sepsis, other illnesses requiring mechanical ventilation, drug-induced muscle disease (such as glucocorticoid-induced myopathy and statin-induced myopathy), genetic diseases that primarily affect skeletal muscle (such as muscular dystrophy, myotonic dystrophy and inclusion body myositis), or autoimmune diseases that affect skeletal muscle (such as polymyositis and dermatomyositis), or muscle wasting from medications such as GLP-1 agonists.

Cachexia is an acquired, accelerated loss of muscle caused by an underlying disease. In some instances, cachexia refers to a loss of body mass that cannot be reversed nutritionally, and is generally associated with an underlying disease, such as cancer, COPD, AIDS, heart failure, and the like. When cachexia is seen in a patient with end-stage cancer, it is called “cancer cachexia”. Cancer cachexia affects the majority of patients with advanced cancer and is associated with a reduction in treatment tolerance, response to therapy, quality of life and duration of survival. It some instances, cancer cachexia is defined as a multifactorial syndrome characterized by an ongoing loss of skeletal muscle mass, with or without loss of fat mass, which cannot be fully reversed by conventional nutritional support and leads to progressive functional impairment. In some cases, skeletal muscle loss appears to be the most significant event in cancer cachexia. In addition, the classification of cancer cachexia suggests that the diagnostic criteria takes into account not only that weight loss is a signal event of the cachectic process but that the initial reserve of the patient should also be considered, such as low BMI or low level of muscularity.

In some embodiments, described herein is a method of treating cachexia-associated atrophy in a non-ischemic atrophied muscle, which comprises administering to the subject a hybrid adeno-associated virus (AAV) comprising a nucleotide sequence encoding an E-selectin, in amount effective to treat the cachexia-associated atrophy in a non-ischemic atrophied muscle.

Sarcopenia is the continuous process of muscle atrophy in the course of regular aging that is characterized by a gradual loss of muscle mass and muscle strength over a span of months and years. A regular aging process means herein an aging process that is not influenced or accelerated by the presence of disorders and diseases which promote skeletomuscular neurodegeneration.

In some embodiments, described herein is a method of treating muscle atrophy associated with or induced by sarcopenia in a subject, which comprises administering to the subject a hybrid adeno-associated virus (AAV) comprising a nucleotide sequence encoding an E-selectin, in amount effective to treat atrophied muscle of the subject, wherein the muscle atrophy is in non-ischemic atrophied muscle.

Disuse-associated muscle atrophy results when a limb is immobilized (e.g., due to a limb or joint fracture or an orthopedic surgery such as a hip or knee replacement surgery). As used herein, “immobilization” or “immobilized” refers to the partial or complete restriction of movement of limbs, muscles, bones, tendons, joints, or any other body parts for an extended period of time (e.g., for 2 days, 3 days, 4 days, 5 days, 6 days, a week, two weeks, or more). In some instances, a period of immobilization includes short periods or instances of unrestrained movement, such as to bathe, to replace an external device, or to adjust an external device. Limb immobilization is optionally carried out by any variety of external devices including, but are not limited to, braces, slings, casts, bandages, and splints (any of which is optionally composed of hard or soft material including but not limited to cloth, gauze, fiberglass, plastic, plaster, or metal), as well as any variety of internal devices including surgically implanted splints, plates, braces, and the like. In the context of limb immobilization, the restriction of movement involves a single joint or multiple joints (e.g., simple joints such as the shoulder joint or hip joint, compound joints such as the radiocarpal joint, and complex joints such as the knee joint, including but not limited to one or more of the following: articulations of the hand, shoulder joints, elbow joints, wrist joints, auxiliary articulations, sternoclavicular joints, vertebral articulations, temporomandibular joints, sacroiliac joints, hip joints, knee joints, and articulations of the foot), a single tendon or ligament or multiple tendons or ligaments (e.g., including but not limited to one or more of the following: the anterior cruciate ligament, the posterior cruciate ligament, rotator cuff tendons, medial collateral ligaments of the elbow and knee, flexor tendons of the hand, lateral ligaments of the ankle, and tendons and ligaments of the jaw or temporomandibular joint), a single bone or multiple bones (e.g., including but not limited to one or more of the following: the skull, mandible, clavicle, ribs, radius, ulna, humorous, pelvis, sacrum, femur, patella, phalanges, carpals, metacarpals, tarsals, metatarsals, fibula, tibia, scapula, and vertebrae), a single muscle or multiple muscles (e.g., including but not limited to one or more of the following: latissimus dorsi, trapezius, deltoid, pectorals, biceps, triceps, external obliques, abdominals, gluteus maximus, hamstrings, quadriceps, gastrocnemius, and diaphragm); a single limb or multiple limbs one or more of the arms and legs)), or the entire skeletal muscle system or portions thereof (e.g., in the case of a full body cast or spica cast).

### Muscle Tears

In another aspect, the disclosed provides methods and compositions for treating a muscle tear in non-ischemic muscle tissue in a subject in need thereof, comprising administering to the subject a hybrid adeno-associated virus (AAV) comprising a nucleotide sequence encoding an E-selectin, in amount effective to treat the muscle tear in the subject.

### Connective Tissue Reattachment

In another aspect, the disclosure provides methods and compositions for improving the outcome of a connective tissue reattachment procedure in a subject in need thereof comprising administering to the subject a hybrid adeno-associated virus (AAV) comprising a nucleotide sequence encoding an E-selectin in an amount effective to improve the outcome of procedure. “Improving the outcome” as used herein refers to an at least 10% (e.g., 10%, 15%, 20%, 30%, 40%, 50%, 60%, 70%, 80%, 90% or more) improvements in the healing time as seen by faster recovery of pain-free limb function and tissue repair as seen by magnetic resonance medical imaging of the injured musculoskeletal body part compared to subjects that were not treated with the AAV.

In some embodiments, the procedure is rotator cuff repair, Achilles tendon repair, patellar-patella tendon repair, medial cruciate ligament (MCL) reconstruction, anterior cruciate ligament (ACL) reconstruction, ulnar collateral ligament (UCL), meniscus repair, or labrum repair. In some embodiments, the connective tissue is a ligament, tendon, meniscus or a labrum.

### E-selectin

E-selectin is a cell adhesion molecule typically expressed on endothelial cells. E-selectin is also known as CD62 antigen-like family member E (CD62E), endothelial-leukocyte adhesion molecule 1 (ELAM-1), and leukocyte-endothelial cell adhesion molecule 2 (LECAM2). In various aspects, the E-selectin is native human E-selection. In this regard, the nucleic acid sequence encoding E-selectin is optionally a nucleic acid sequence encoding the human E-selectin protein (i.e., the E-selectin protein of SEQ ID NO: 1, which corresponds to Accession no. AAQ67702, NP_000441.2). In exemplary aspects, the nucleic acid sequence encodes the mature form of human E-selectin and does not contain a signal peptide MIASQFLSALTLVLLIKESGA (SEQ ID NO: 7). In exemplary aspects, the nucleic acid sequence encodes the mature form of human E-selectin of SEQ ID NO: 8. In various embodiments, the nucleic acid sequence encodes a protein that shares at least 65% (e.g., at least 75%, at least 80%, at least 85%, at least 90%, at least 95%, at least 96%, at least 97%, at least 98%, or 99%) amino acid sequence identity with SEQ ID NO: 1 and demonstrates at least one activity associated with native E-selectin, such as mediating EC-EPC adhesion or promoting accumulation of blood leukocytes at sites of inflammation. In various aspects, the nucleic acid sequence encoding E-selectin is set forth in SEQ ID NO: 2, which corresponds to Accession no. NM_000450. It will be appreciated that a nucleic acid encoding an allelic variant and homolog of human E-selectin is also contemplated. In various embodiments, the nucleic acid sequence is at least 65% (e.g., at least 75%, at least 80%, at least 85%, at least 90%, at least 95%, at least 96%, at least 97%, at least 98%, or 99%) identical to SEQ ID NO: 2. If desired, non-human, mammalian E-selectin also may be used; the amino acid sequence of mouse E-selectin (GenBank Accession No. AAA37577.1), rat E-selectin (GenBank Accession No. AAA41113.1), canine E-selectin (GenBank Accession No. AAA30843.1), and sheep E-selectin (GenBank Accession No. NP_001009749.1) are provided as SEQ ID NOs: 3-6, respectively.

As used herein, “at least 90% identity” and similar terms encompass any integer from, e.g., 90% to 100%, such as 90%, 91%, 92%, 93%, 94%, 95%, 96%, 97%, 98% or 99% and the like. Also, the term “at least [percentage] identity” encompasses any percentage that is greater than or equal to the number of identical nucleotides or amino acids divided by the total number of nucleotides or amino acids ([at least percentage identity]×[number of identical nucleotides or amino acids]/[total number of nucleotides or amino acids]). The calculation of percent identity of aligned amino acids (or nucleotides) of two or more sequences is well understood in the art and is determined conventionally using known computer programs. For example, alignment of two or more sequences to determine percent sequence identity is optionally performed using the algorithm described by Altschul et al. (Nucleic Acids Res., 25:3389-402 (1997)) as incorporated into BLAST (basic local alignment search tool) programs, available on the National Center for Biotechnology Information website.

Variant E-selectin proteins that differ from SEQ ID NO: 1 can be generated by making nucleotide substitutions that cause changes in the encoded polypeptide. Examples of substitutions are those that cause changes in (a) the structure of the polypeptide backbone; (b) the charge or hydrophobicity of the polypeptide; or (c) the bulk of an amino acid side chain. In various aspects, the variant E-selectin comprises one or more conservative substitutions, i.e., at least one amino acid of the protein is substituted with another amino acid having similar characteristics.

### Adeno-Associated Virus

In various embodiments, the method comprises administering to a subject an effective amount of a hybrid adeno-associated virus (AAV) comprising a nucleotide sequence encoding an E-selectin. By “hybrid AAV” is meant an AAV comprising portions of at least two AAV serotypes. In exemplary aspects, the hybrid AAV is not naturally-occurring and is engineered to comprise portions of AAV from two different AAV serotypes. “Hybrid AAV” are synonymous with AAV hybrid serotypes as described in Choi et al., Current Gene Ther 5(3): 299-310 (2005) and Wu et al., Mol Ther. 14(3):316-27 (2006). In exemplary aspects, the hybrid AAV comprises AAV2 ITRs in the viral genome, which is packaged in a capsid from an AAV other than serotype 2. The AAV mediates E-selectin production in target cells.

In various aspects, the methods described herein comprise administering to the subject a cell comprising an AAV comprising viral genome comprising a nucleotide sequence encoding an E-selectin and AAV2 ITRs, which is packaged into an AAV2 capsid.

AAV is a DNA virus not known to cause human disease, making it a desirable gene therapy options. The AAV genome is comprised of two genes, rep and cap, flanked by inverted terminal repeats (ITRs), which contain recognition signals for DNA replication and viral packaging. AAV requires co-infection with a helper virus (i.e., an adenovirus or a herpes virus), or expression of helper genes, for efficient replication. AAV vectors used for administration of a therapeutic nucleic acid typically have a majority of the parental genome deleted, such that only the ITRs remain, although this is not required. Delivering the AAV rep protein enables integration of the AAV vector comprising AAV ITRs into a specific region of genome, if desired. Host cells comprising an integrated AAV genome show no change in cell growth or morphology. As such, prolonged expression of therapeutic factors from AAV vectors can be useful in treating persistent and chronic diseases. The AAV for use in the context of the disclosure is based on AAV type 2, and the viral genome delivered to the subject or cell comprises AAV2 ITRs. Other AAV serotypes include AAV type 1, AAV type 3 (including types 3A and 3B), AAV type 4, AAV type 5, AAV type 6, AAV type 7, AAV type 8, AAV type 9, AAV type 10, or AAV type 11. The genomic sequences of AAV, as well as the sequences of the ITRs, Rep proteins, and capsid subunits are known in the art. See, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

In various aspects, the AAV comprises a viral genome lacking all or part of the native AAV genome. For example, the AAV genome lacks all native AAV protein coding sequences, but retains the AAV ITRs (e.g., AAV2 ITRs), and further comprises the nucleic acid sequence encoding E-selectin.

The viral genome comprising the nucleic acid sequence and AAV ITRs can be incorporated into an virion (i.e., packaged into a viral capsid) to facilitate introduction of the genome into a cell. AAV capsid proteins compose the exterior, non-nucleic acid portion of the virion and are encoded by the AAV cap gene. The cap gene encodes three viral coat proteins, VP1, VP2 and VP3, which are required for virion assembly. The construction of AAV virions is described in, e.g., U.S. Pat. Nos. 5,173,414; 5,139,941; 5,863,541; 5,869,305; 6,057,152; and U.S. Pat. No. 6,376,237; Rabinowitz et al., J. Virol. 76:791-801, 2002; and Bowles et al., J. Virol. 77:423-432, 2003.

Optionally, the AAV genome of one serotype is packaged into a capsid of the same serotype, e.g., an AAV genome comprising AAV2 ITRs is packaged into an AAV2 capsid. Alternatively, an AAV genome of one serotype is packaged into a capsid of a second, different serotype. For example, in embodiments, the AAV genome comprising AAV2 ITRs is packaged into a capsid derived from a serotype other AAV2. Such AAV vectors are termed “pseudotyped” AAV or “hybrid” AAV. The AAV2 viral genome (comprising the nucleic acid sequence encoding E-selectin and AAV2 ITRs) is optionally packaged into a capsid from AAV type 1, AAV type 3 (including types 3A and 3B), AAV type 4, AAV type 5, AAV type 6, AAV type 7, AAV type 8, AAV type 9, AAV type 10, or AAV type 11. In various aspects, the AAV2 viral genome is packaged into an AAV8 capsid (AAV2/8) or AAV9 capsid (AAV2/9). Techniques involving the construction and use of pseudotyped AAV are further described in, e.g., Duan et al., J. Virol, 75:7662-7671, 2001; Halbert et al., J. Virol, 74:1524-1532, 2000; Zolotukhin et al, Methods, 28: 158-167, 2002; and Auricchio et al, Hum. Molec. Genet. 10:3075-3081, 2001.

Optionally, the virus capsid (i.e., particle surface) is modified to adjust viral tropism. For example, components of the capsid can be modified to, e.g., expand the types of cells transduced by the resulting vector, avoid (in whole or in part) transduction of undesired cell types, or improve transduction efficiency of desired cell types (e.g., by incorporating a ligand for a cell surface receptor on desired cell type). Transduction efficiency is generally determined by reference to a control (i.e., an unmodified, matched viral vector). Improvements in transduction efficiency can result in, e.g., at least about 25%, 50%, 60%, 70%, 80%, 85%, 90%, 95%, 100% improvement in transduction rate of a given cell type. If desired, the capsid can be modified such that it does not efficiently transduce non-target tissues, such as liver or germ cells (e.g., 50% or less, 30% or less, 20% or less, 10% or less, 5% or less of the level of transduction of desired target tissue(s)). The construction and characterization of AAV capsid mutants including insertion mutants, alanine screening mutants, and epitope tag mutants is described in Wu et al, J. Virol. 74:8635-45, 2000. Other AAV that can be used in methods described herein include capsid hybrids that are generated by molecular breeding of viruses, as well as by exon shuffling. See Soong et al, Nat. Genet. 25:436-439, 2000; and Kolman and Stemmer Nat. Biotechnol 19:423-428, 2001.

Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001, all of which are hereby incorporated by reference, particularly with respect to the discussion of AAV production. Methods for using AAV vectors also are discussed, for example, in Tal, J., J. Biomed. Sci. 7:279-291, 2000 and Monahan and Samulski, Gene delivery 7:24-30, 2000.

Expression vectors, such as AAV vectors, typically contain a variety of nucleic acid sequences necessary for the transcription and translation of an operably linked coding sequence. For example, an expression vector can comprise origins of replication, polyadenylation signals, internal ribosome entry sites (IRES), promoters, enhancers, and the like. The AAV vector of the disclosure preferably comprises a promoter operably linked to the E-selectin coding sequence. “Operably linked” means that a control sequence, such as a promoter, is in a correct location and orientation in relation to another nucleic acid sequence to exert its effect (e.g., initiation of transcription) on the nucleic acid sequence. A promoter can be native or non-native to the nucleic acid sequence to which it is operably linked and native or non-native to a particular target cell type, and the promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Examples of constitutive mammalian promoters include various housekeeping gene promoters, as exemplified by the β-actin promoter. Inducible promoters and/or regulatory elements are also contemplated for use in the methods described herein. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. Tissue-specific promoters and/or regulatory elements are useful in certain embodiments of the methods described herein. Examples of such promoters include, but are not limited to, the Tie-2 or KDR promoter.

### Administration of Cells Producing E-Selectin

In some embodiments, the methods described herein comprise administering to the subject a cell comprising an AAV comprising a nucleotide sequence encoding an E-selectin (optionally comprising AAV2 ITRs). The AAV produces E-selectin in the cell. In various aspects, the AAV2 genome is packaged into an AAV2 capsid, although a pseudotyped AAV also may be employed (e.g., the AAV genome comprising AAV2 ITRs may be packaged into a non-AAV2 capsid in various embodiments, as described further herein). The cell is, in various embodiments, a stem cell, such as a mesenchymal stem cell (MSC), a bone marrow (BM)-derived progenitor cell, a fibroblast mature or progenitor cell type, a skeletal muscle mature or progenitor cell type, or an endothelial progenitor cell (EPC). The cell may be isolated from the subject (i.e., autologous) or collected from a different donor (i.e., allogeneic). “Bone marrow-derived progenitor cells” and “BM-derived progenitor cells” mean progenitor cells that come from a bone marrow stem cell lineage. The cell also may be a mesenchymal stem cell (MSC), embryonic-like cells found in bone marrow that are capable of osteogenic, myogenic, adipogenic and chondrogenic differentiation.

In various aspects, the cell is an endothelial progenitor cell (EPC). By the “progenitor cell” or “endothelial progenitor cells” or “EPC” is meant any somatic cell which has the capacity to generate fully differentiated, functional progeny by differentiation and proliferation. In another embodiment, progenitor cells include progenitors from any tissue or organ system, including, but not limited to, blood, nerve, muscle, skin, gut, bone, kidney, liver, pancreas, thymus, and the like. Progenitor cells are distinguished from “differentiated cells,” which are cells which may or may not have the capacity to proliferate, i.e., self-replicate, but which are unable to undergo further differentiation to a different cell type under normal physiological conditions. Progenitor cells are further distinguished from abnormal cells such as cancer cells, especially leukemia cells, which proliferate (self-replicate) but which generally do not further differentiate, despite appearing to be immature or undifferentiated.

“Totipotent” cells are uncommitted progenitor cells, such as embryonic stem cells, i.e., both necessary and sufficient for generating all types of mature cells. Progenitor cells which retain a capacity to generate all pancreatic cell lineages but which cannot self-renew are termed “pluripotent.” In another embodiment, cells which can produce some but not all endothelial lineages and cannot self-renew are termed “multipotent.”

Techniques for isolating donor stem cells and transplantation of such isolated cells are known in the art. For example, target tissue or cells (e.g., BM-derived EPCs) are harvested from a host and exposed to the AAV virions described herein under conditions that promote infection, thereby introducing the E-selectin-encoding nucleic acid into the cells. These genetically modified cells are then be transplanted into the subject. Several approaches may be used for the introduction of cells into the subject, including intravenous injection, intraperitoneal injection, or in situ injection into target tissue. Microencapsulation of cells transduced or infected with AAV also is contemplated. Both autologous and allogeneic cell transplantation are contemplated in the context of the method of the disclosure.

### Other Methods of Use

The present disclosure evaluated the effects of E-selectin gene therapy on skeletal muscle recovery, specifically focusing on exercise performance and myofiber regeneration. C57BL/6J mice were treated with intramuscular E-selectin/adeno-associated virus serotype 2/2 gene therapy (E-sel/AAV) or LacZ/AAV2/2 (LacZ/AAV) as control and then subjected to femoral artery coagulation. Recovery of hindlimb perfusion was assessed by laser Doppler perfusion imaging and muscle function by treadmill exhaustion and grip strength testing. After three postoperative weeks, hindlimb muscle was harvested for immunofluorescence analysis. At all postoperative time points, mice treated with E-sel/AAV had improved hindlimb perfusion and exercise capacity. E-sel/AAV gene therapy also increased co-expression of MyoD and Ki-67 in skeletal muscle progenitors and the proportion of Myh7⁺ myofibers.

In aspects, the present disclosure demonstrates that in addition to improving reperfusion, intramuscular E-sel/AAV gene therapy enhances regeneration of ischemic skeletal muscle with corresponding benefit on exercise performance. These results highlight a role for E-sel/AAV gene therapy as a nonsurgical adjunct in patients with life-limiting PAD.

In aspects, the present disclosure provides E-selectin/AAV gene therapy for improving myogenesis (muscle regeneration), decreased atrophy, and functional recovery of skeletal muscle in ischemic limbs.

In aspects, the present disclosure provides E-selectin/AAV gene therapy for improving myogenesis (muscle regeneration), and functional recovery of skeletal muscle in subjects suffering from ischemia-reperfusion swelling and compartment syndrome in an acute ischemic limb. In some embodiments, acute ischemia is caused by emboli, crush injury, or extreme exercise in a subject.

E-selectin/AAV gene therapy increases activation of myogenic precursors in ischemic limbs, and promotes type 1 myofiber regeneration based on observations from mouse limb ischemia model treated with E-selectin/AAV gene therapy administered via intramuscular injection.

In aspects, the present disclosure provides methods and compositions for directing AAV2 to deliver E-selectin to skeletal muscle to improve perfusion and exercise performance.

### Formulations, Dose, Administration Regimens

In various aspects, the AAV or the cell is provided in a composition (e.g., a pharmaceutical composition) comprising a physiologically-acceptable (i.e., pharmacologically-acceptable) carrier, buffer, excipient, or diluent. Any suitable physiologically-acceptable (e.g., pharmaceutically acceptable) carrier can be used within the context of the disclosure, and such carriers are well known in the art. The choice of carrier will be determined, in part, by the particular site to which the composition is to be administered and the particular method used to administer the composition. The composition also can comprise agents which, for instance, facilitate uptake of the AAV into host cells. Suitable composition formulations include aqueous and non-aqueous solutions, isotonic sterile solutions, which can contain antioxidants, buffers, bacteriostats, and solutes that render the formulation isotonic with the blood, and aqueous and non-aqueous sterile suspensions that can include suspending agents, solubilizers, thickening agents, stabilizers, and preservatives.

The composition may be formulated for topical administration (e.g., in the form of aerosol, cream, foam, gel, liquid, ointment, paste, powder, shampoo, spray, patch, disk, or dressing). A “patch” typically includes at least the compositions provided herein and a covering layer, such that, the patch can be placed over an area of skin to be treated. The patch can be designed to maximize delivery of the compositions provided herein through the stratum corneum and into the epidermis or dermis, reduce lag time, promote uniform absorption, and reduce mechanical rub-off.

The composition can be presented in unit-dose or multi-dose sealed containers, such as ampules and vials, and can be stored in a freeze-dried (lyophilized) condition requiring only the addition of the sterile liquid carrier, for example, water, immediately prior to use. A composition comprising AAV or cells comprising AAV is, in one aspect, placed within containers, along with packaging material that provides instructions regarding the use of the composition (i.e., in a kit). Generally, such instructions include a tangible expression describing the reagent concentration, as well as, in certain embodiments, relative amounts of excipient ingredients or diluents (e.g., water, saline or PBS) that may be necessary to reconstitute the composition.

The AAV or cell is administered in an amount and at a location sufficient to provide some improvement or benefit to the subject, e.g., promote muscle regeneration in atrophied muscle tissue. Depending on the circumstances, a composition comprising the AAV or cell is applied or instilled into body cavities, applied directly to target tissue, and/or introduced into circulation. For example, in various circumstances, it will be desirable to deliver the composition by intravenous, intraperitoneal, intracerebral (intra-parenchymal), intramuscular, intra-ocular, intraarterial, intraportal, intralesional, intramedullary, intrathecal, intraventricular, intradermal, intraarticular, intraneuronal, intraganglion, periganglion, transdermal, subcutaneous, intranasal, inhalation (e.g., upper and/or lower airways), enteral, epidural, urethral, vaginal, or rectal means. If desired, the AAV or cell is administered regionally via intramuscular, transdermal, or subcutaneous administration, or intraarterial or intravenous administration feeding the region of interest. In some embodiments, the AAV or cell is intramuscularly administered to non-ischemic atrophied skeletal muscle.

In some embodiments, the AAV or cell is administered intramuscularly to an injured connective tissue attachment of the muscle in the subject. In some embodiments, the AAV or cell is administered intra-joint within the synovial fluid to the injured connective tissue attachment of the muscle in the subject. In some embodiments, the AAV or cell is administered systemically to the subject with multiple injured connective tissue attachments to muscles. In some embodiments, the AAV or cell is administered topically to the subject with multiple injured connective tissue attachments to exposed muscles.

A particular administration regimen for a particular subject will depend, in part, upon the amount of therapeutic administered, the route of administration, and the cause and extent of any side effects. The amount administered to a subject (e.g., a mammal, such as a human) in accordance with the disclosure should be sufficient to affect the desired response over a reasonable time frame.

Exemplary doses of viral particles in genomic equivalent titers of 10⁴-10¹⁵ transducing units (e.g., 10⁷-10¹² transducing units), or at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, or at least about 10¹⁵ transducing units (e.g., at least about 10⁷, at least about 10⁸, at least about 10⁹, at least about 10¹⁰, at least about 10¹¹, at least about 10¹², at least about 10¹³ or at least about 10¹⁴ transducing units, such as about 10¹⁰ or 10¹² transducing units). In exemplary aspects, the dose of viral particles (VP) per in vitro transduced cell is within about 10³ to about 10¹². In some aspects, the dose of viral particles per in vitro transduced cell is within about 10⁴ to about 10⁸ or about 10⁴ to about 10⁶. For example, the dose of viral particles per in vitro transduced cell is 10⁵ VP/cell. Some conditions require prolonged treatment, which may or may not entail multiple administrations over time.

In some embodiments, the dose of the AAV administered to the subject (e.g., via intramuscular injection) is about 50 to about 5000 μl hybrid AAV, wherein the concentration of the hybrid AAV is within about 10⁸ or 10¹⁶ VP/ml. In some embodiments, the dose of the hybrid AAV administered to the subject (e.g., via intramuscular injection) is about 50 to about 500 μl hybrid AAV, wherein the concentration of the hybrid AAV is within about 10¹⁰ or 10¹⁴ VP/ml. In some embodiments, the dose of the hybrid AAV administered to the subject (e.g., via intramuscular injection) is about 75 to about 200 μl hybrid AAV, wherein the concentration of the hybrid AAV is about 10¹² VP/ml.

When appropriate, the AAV or cell is administered in combination with other substances (e.g., therapeutics) and/or other therapeutic modalities to achieve an additional (or augmented) biological effect. This aspect includes concurrent administration (i.e., substantially simultaneous administration) and non-concurrent administration (i.e., administration at different times, in any order, whether overlapping or not) of the AAV or cell and one or more additionally suitable agents(s). It will be appreciated that different components are, in certain aspects, administered in the same or in separate compositions, and by the same or different routes of administration.

In some embodiments, the AAV or cell is administered separately, sequentially or simultaneously in combination with one or more agents useful for treating the symptoms or causes of muscle atrophy. Representative co-therapies include, but are not limited to, physical therapy, functional electrical stimulation (FES), and ultrasound therapy.

In some embodiments, the AAV or cell is administered separately, sequentially or simultaneously in combination with one or more agents useful for pain management. Examples of further agents include, but are not limited to, an opioid analgesic (e.g., morphine, hydromorphone, oxymorphone, fentanyl, codeine, dihydrocodeine, oxycodone, or hydrocodone); a nonsteroidal anti-inflammatory drug (NSAID) (e.g., aspirin, diclofenac, ibuprofen, naproxen, oxaprozin, or cyclooxygenase-2 (COX-2) inhibitor); a sedative (e.g., a barbiturate sedative); an anesthetic; and a corticosteroid (e.g., dexamethasone).

In instances where it is desired to promote recruitment of bone marrow (BM)-derived stem cells from the BM to a non-BM compartment (e.g., a target tissue), an agent capable of promoting recruitment of BM-derived progenitor cells also is provided to the subject, either as part of the composition or separate as part of a treatment regimen. A number of such agents are known and include, e.g., integrins, the selectin family of adhesion molecules, VCAM-I, and colony stimulating factors. Suitable agents are further described in, e.g., International Patent Publication WO 00/50048.

### Other Viral Vectors

It is further contemplated that the viral vector for delivering the E-selectin is retroviral vector other than an adeno-associated viral vector. Exemplary other retroviral vectors include, but are not limited to, lentiviral vectors, an adenoviral vector, a vaccinia viral vector, or a modified vaccinia Ankara (MVA) viral vector. Constructs as described above with respect to the E-selectin can also be made in these other viral vectors. For example, the present disclosure provides recombinant vectors comprising nucleic acid encoding E-selectin, wherein the recombinant vector is a lentiviral vector, an adenoviral vector a vaccinia viral vector or a MVA viral vector. In some embodiments, the recombinant vector is a lentiviral vector.

Retroviruses are enveloped RNA viruses that are capable of infecting animal cells, and that utilize the enzyme reverse transcriptase in the early stages of infection to generate a DNA copy from their RNA genome, which is then typically integrated into the host genome. Examples of retroviral vectors Moloney murine leukemia virus (MLV)-derived vectors, retroviral vectors based on a Murine Stem Cell Virus, which provides long-term stable expression in target cells such as hematopoietic precursor cells and their differentiated progeny (see, e.g., Hawley et al., PNAS USA 93:10297-10302, 1996; Keller et al., Blood 92:877-887, 1998), hybrid vectors (see, e.g., Choi, et al., Stem Cells 19:236-246, 2001), and complex retrovirus-derived vectors, such as lentiviral vectors.

Examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al., Hum Gene Ther. 10:1479-1489, 1999; Case et al., PNAS USA 96:2988-2993, 1999; Uchida et al., PNAS USA 95:11939-11944, 1998; Miyoshi et al., Science 283:682-686, 1999; Sutton et al., J Virol 72:5781-5788, 1998; and Frecha et al., Blood. 112:4843-52, 2008).

Adenoviral vectors, methods for construction thereof and methods for propagating thereof, are well known in the art and are described in, for example, U.S. Pat. Nos. 9,125,870, 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication,” M. S. Horwitz, “Adenoviruses,” Chapters 67 and 68, respectively, in Virology, B. N. Fields et al., eds., 3d ed., Raven Press, Ltd., New York (1996).

Vaccinia viruses have been used for decades as vectors for foreign antigens (Smith et al., Biotechnology and Genetic Engineering Reviews 2. 383-407 [1984]). Methods of inserting foreign DNA into vaccinia virus is well-known to those in the field of vaccine development and protein engineering.

Modified Vaccinia Ankara (MVA) virus is related to vaccinia virus. MVA was engineered for use as a viral vector for recombinant gene expression or as a recombinant vaccine (Sutter, G. et al. [1994], Vaccine 12: 1032-40). Modified MVA for use as vaccines or other viral vector are described in U.S. Pat. Nos. 6,913,752, 6,960,345, 9,133,478 and 9,463,238.

Construction of viral vectors involves the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DNA, 2d ed., Scientific American Books (1992), and Ausubel et al., Current Protocols in Molecular Biology, Wiley Interscience Publishers, NY (1995), and other references mentioned herein.

Unless defined otherwise, all technical and scientific terms used herein have the same meaning as is commonly understood by one of skill in the art to which this disclosure belongs. All patents and publications referred to herein are incorporated by reference in their entireties.

This invention is further illustrated by the following non-limiting examples.

### Example

Hereinafter, the present disclosure will be described in further detail with reference to examples. These examples are illustrative purposes only and are not to be construed to limit the scope of the present disclosure. In addition, various modifications and variations can be made without departing from the technical scope of the present disclosure.

### Materials and Methods

### Production of Adeno-Associated Virus Vectors

Murine E-selectin and LacZgenes were inserted into multiple cloning sites in the pZac vector. After confirmation by Sanger sequencing, E-selectin/pZac and LacZ/pZac plasmids were sent to the University of North Carolina Gene Therapy Vector Core where AAV serotype 2/2 was prepared by three-plasmid transfection into HEK293 cells [25]. Quality assurance and control testing was performed by polymerase chain reaction (PCR) quantification of genomes and infectivity titer.

### Gene Therapy Administration

Animal experiments were performed in C57BL/6J male and female mice (000664, Jackson Laboratory, Bar Harbor, ME) aged 10-12 weeks old. All protocols were approved by the University of Miami Institutional Animal Care and Use Committee (22-096). To account for the lag time between AAV injections and tissue transgene expression, gene therapy was administered 4 and 2 days prior to and on the day of surgery. Total dose per mouse was 1×10¹¹ viral genome divided across the 3 days and diluted in 100 μL phosphate-buffered saline (PBS) each day. Mice were anesthetized with inhaled isoflurane 1.5-2% and oxygen at 2 L/min and administered intramuscular (IM) injection of either E-sel/AAV or LacZ/AAV (N=20 per group, 10 females per group) into five sites in the left adductor group (2), lateral thigh (1), and medial (1) and lateral (1) gastrocnemius.

### Induction of Hindlimb Ischemia

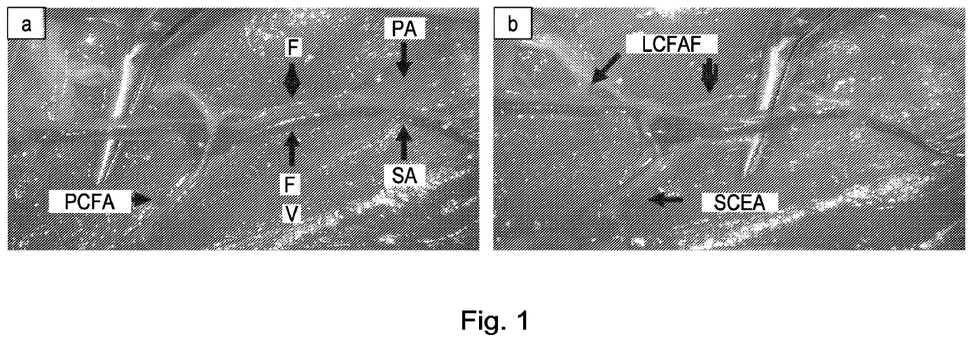

Hindlimb ischemia was induced according to previously described protocol [26]. Mice were anesthetized by intraperitoneal (IP) injection of ketamine (80 mg/kg) and xylazine (5 mg/kg). After hair removal, the left groin was prepared with chlorohexidine. A 1 cm incision was made in the left groin and the inguinal fat dissected from the inguinal ligament. The femoral sheath was entered and the femoral nerve isolated from the femoral vessels. The femoral artery and vein were coagulated with an electrocautery device just proximal to the lateral circumflex femoral artery (FIG. 1A) and just proximal to the saphenopopliteal bifurcation (FIG. 1B). Hemostasis was obtained and the wound was closed with 5-0 absorbable suture.

### Laser Doppler Perfusion Imaging

Hindlimb perfusion was measured using a moorLDI laser Doppler perfusion imaging (LDPI) device and quantified in version 5 software (Moor Instruments, Wilmington, DE). To acquire images, mice (N=20 per group) were anesthetized with inhaled isoflurane 1.5-2% and oxygen at 2 L/min and placed in prone position on a black foam mat. Body temperature was maintained with a heating pad. After scanning, perfusion index was calculated as the ratio of mean flux values from the left/ischemic relative to right/non-ischemic hindlimb.

### Grip Strength Testing

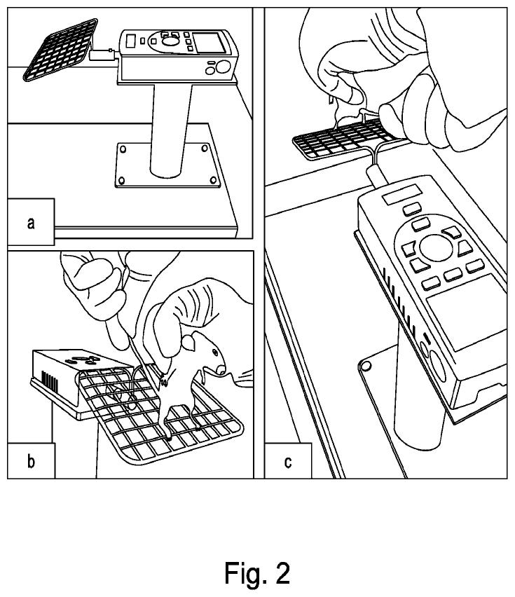

Grip strength was measured using a grip strength meter (Columbus Instruments, Columbus, OH) fitted with a mesh grid assembly and set to peak compression mode (FIG. 2A). Mice (N=20 per group) were held by the dorsal skin and placed on the grid assembly such that only the hindlimbs grasped the grid (FIG. 2B). The animal was then gently pulled backwards towards the grip strength meter and the maximal compression force recorded in gram-force (gf) units (FIG. 2C). Three separate readings were obtained during each session. Results are reported as the mean of three readings and the best reading normalized to body weight at time of testing.

### Treadmill Exhaustion Testing

Mice (N=8 per group) were trained to run on an Exer 3/6 treadmill (Columbus Instruments) during 4 sessions across the 2 weeks prior to surgery. The treadmill was set at a 10° incline with shocks at 1 Hz. For training sessions, mice walked on the treadmill at a speed of 10 m/min for 10 minutes and then 15 m/min for 5 minutes. For exhaustion testing, mice were allowed to warm up with the treadmill speed set at 5 m/min and then ramped up by 1 m/min². Distance recording was started when speed reached 10 m/min. After 5 minutes, treadmill speed was increased to 15 m/min, and then by 3 m/min every 5 minutes until maximum speed of 30 m/min. Exhaustion was defined as 40 shocks after which they were disabled, and total walking distance was recorded.

### Immunofluorescence Assays

Mice were euthanized on POD 21 for harvesting of the left and right adductor and gastrocnemius muscles. Tissue samples were fixed in 10% formalin, embedded in paraffin, and sectioned. Slides were deparaffinized per standard protocol and antigen retrieval was performed in EDTA buffer (pH 9.0) at 120° C. for 10 minutes. Slides were washed in distilled water and permeabilized with 0.25% Triton-X100 TBS for 15 minutes. Tissue was incubated with Protein Block (ab64226, Abcam, Cambridge, United Kingdom) for 1 hour. Slides were then incubated overnight at 4° C. with primary antibodies (5 μg/mL) for E-selectin (148802, BioLegend, San Diego, CA), MyoD (NBP1-54153, Novus Biologicals, Littleton, CO), Ki-67 (SC-7846, Santa Cruz Biotechnology, Dallas, TX), laminin (NBP2-44751, Novus), and Myh7 (NBP2-94079, Novus) followed by Alexa Fluor 488 donkey anti-rabbit IgG (A21206, Invitrogen, Waltham, MA), Alexa Fluor 488 goat anti-mouse (A11029), Alexa Fluor 594 chicken anti-goat IgG (A21468, Invitrogen), or Alexa Fluor 594 goat anti-rat IgG (A11007, Invitrogen) as appropriate (2 μg/mL). Slides were imaged at 20× magnification with a Zeiss Axio Observer inverted microscope (ZEISS, Oberkochen, Germany). For each stain, a blinded observer acquired at least 4 images from 4 sections per mouse (N=5 per group) and performed cell counting in Fiji.

### PCR Quantification of E-Sel Transgene Expression

Muscle harvested on POD 21 was homogenized in TRIzol reagent (15596018, Invitrogen/Thermo Fisher Scientific). Total RNA was extracted and reverse transcribed using RT² First Strand Kit (Qiagen, Venlo, Netherlands). Real-time reverse transcription quantitative PCR (RT-qPCR) was performed using RT² SYBR Green qPCR Mastermix (330500, Qiagen) and primers for E-sel (Sele, NM_011345, assay ID Mm.PT.58.11296882, primers 5′-GTCATCCTGTAACTTCACCTGT-3′ and 5′-CGCAGATAAGGCTTCACAC-3′) and Rplp0 (NM_007475, assay ID Mm.PT.58.43894205, primers 5′-TTATAACCCTGAAGTGCTCGAC-3′ and 5′-CGCTTGTACCCATTGATGATG-3′) as housekeeping gene (Integrated DNA Technologies, Coralville, IA). Assays were performed in duplicate (N=4 per group) and analyzed using the ΔCt method (2^{-ΔΔCt}) method.

### Statistics

Statistical analyses were performed using GraphPad Prism (version 9.0.1, GraphPad Software, San Diego, CA). All continuous data were normally distributed by Shapiro-Wilk test and compared using Student's t-test. Data are presented as mean±standard error (SEM) with statistical significance set as P<0.05.X

### Results

### E-Sel/AAV Induces High-Level Transgene Expression in Skeletal Muscle

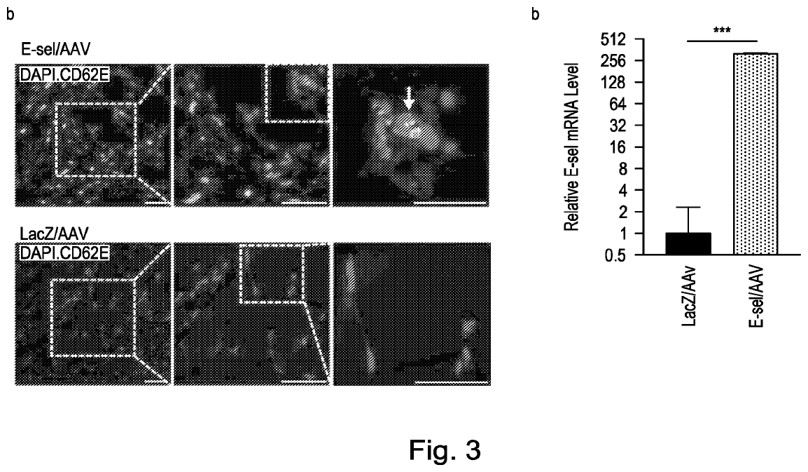

To account for the lag time between AAV injection and tissue transgene expression, which normally takes 2-4 days, gene therapy was administered 4 and 2 days preoperatively and immediately prior to surgery. A total of 1×10¹¹ viral genome divided across 3 doses of either E-sel/AAV or LacZ/AAV control vector was administered to 5 sites in the left thigh adductor and calf muscles. Hindlimb ischemia was then induced by left femoral artery and vein coagulation. Expression of E-selectin after treatment with E-sel/AAV was assessed by immunofluorescence (FIG. 3A) and qRT-PCR (FIG. 3B). In muscle treated with E-sel/AAV, E-selectin was primarily concentrated in the space between muscle fibers and in capillaries. Moreover, E-sel mRNA levels were 322-fold higher in ischemic muscle three after treatment with E-sel/AAV compared to LacZ/AAV, indicating high-level and durable transgene expression with this vector.

### E-Sel/AAV Improves Reperfusion of Ischemic Hindlimb

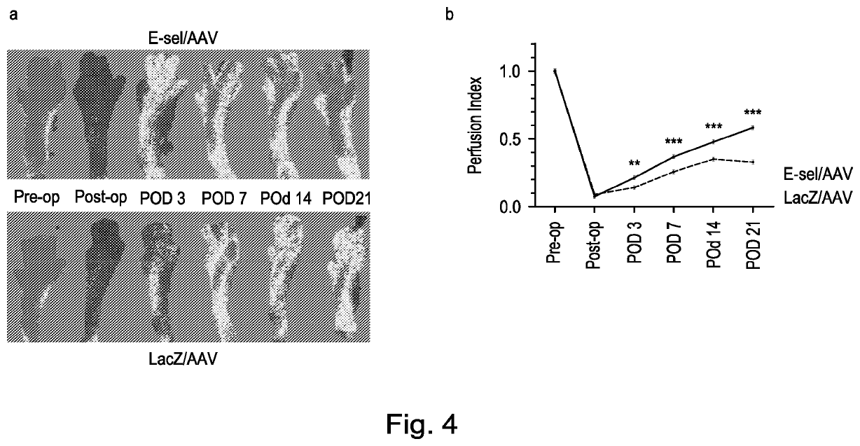

After femoral artery coagulation, both E-sel/AAV- and LacZ/AAV-treated mice experienced a similar reduction in hindlimb perfusion (0.08±0.01 vs 0.08±0.01, P=0.64) (FIG. 4a,b). Perfusion then progressively improved in both groups but was significantly enhanced by E-sel/AAV starting on POD 3 (0.21±0.02 vs 0.14±0.01, P=0.002) and at all time points through POD 21 (0.58±0.02 vs 0.33±0.02, P<0.001).

### E-Sel/AAV Enhances Recovery of Ischemic Hindlimb Grip Strength and Exercise Capacity

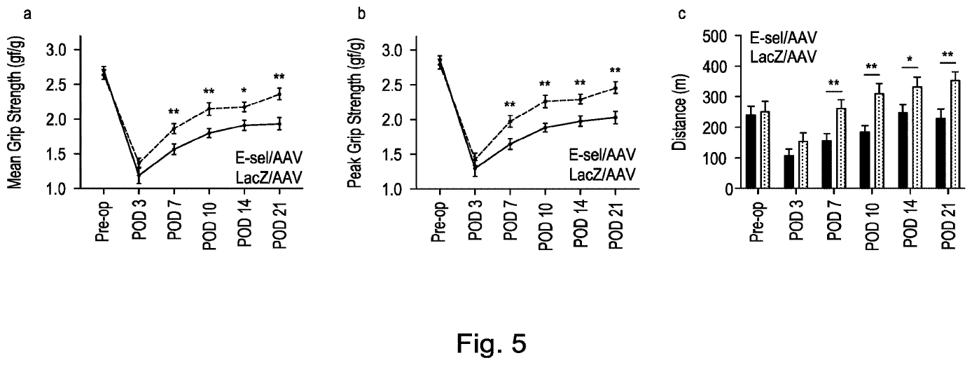

To assess functional recovery, both hindlimb grip strength and aerobic exercise capacity were tested by treadmill exhaustion. In both groups, hindlimb grip strength was acutely impaired after femoral artery coagulation and then gradually recovered in parallel with reperfusion. However, starting on POD 7 (mean grip strength 1.89±0.08 vs 1.57±0.07 gf/g, P=0.009; peak grip strength 1.97±0.08 vs 1.64±0.08 gf/g, P=0.006) and through POD 21 (mean grip strength 2.36±0.08 vs 1.93±0.09 gf/g, P=0.001; peak grip strength 2.45±0.08 vs 2.03±0.09, P=0.001), both mean and peak grip strengths were significantly greater in mice treated with E-sel/AAV compared to LacZ/AAV control vector (FIGS. 5A and 5B). Similarly, recovery of exercise capacity on treadmill exhaustion testing was improved in E-sel/AAV-treated mice compared to controls starting on POD 7 (264±26 vs 157±23 m, P=0.009) and through POD 21 (354±27 vs 232+30 m, P=0.009) (FIG. 5C).

### E-Sel/AAV Increases Activation of Myogenic Precursors

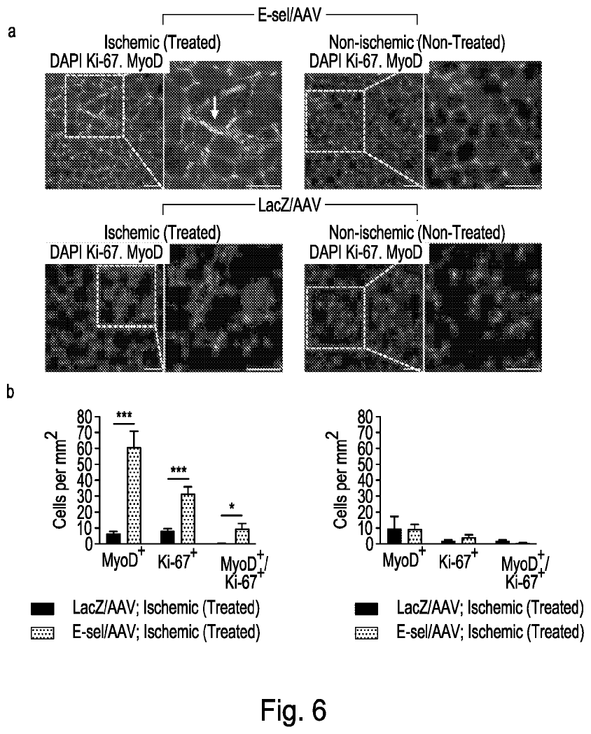

To determine whether treatment with E-sel/AAV affected the activation of skeletal muscle precursors, immunofluorescence staining for the myogenic differentiation marker MyoD and proliferation marker Ki-67 was performed (FIG. 6A). On POD 21, ischemic calf muscle treated with E-sel/AAV demonstrated an increased number of MyoD⁺ cells compared to that treated with LacZ/AAV control vector (61.0±9.9 vs 6.2±1.6 cells/mm², P<0.001) (FIG. 6B). Similarly, there was an increased number of Ki-67⁺ cells in the E-sel/AAV-treated muscle compared to control (31.8±4.3 vs 8.0±1.4 cells/mm², P<0.001). To compare the number of proliferating myogenic precursors, the number of cells co-staining for both MyoD and Ki-67 were counted. As with the individual stains, there was also a greater number of MyoD⁺/Ki-67⁺ cells in ischemic muscle treated with E-sel/AAV compared to LacZ/AAV control vector (9.4±3.6 vs 0.1±0.1 cells/mm², P=0.027). In contrast, there were few cells expressing MyoD (8.9±2.9 vs 9.0±8.0 cells/mm², P=0.99), Ki-67 (3.5±1.6 vs 1.3±0.7 cells/mm², P=0.29), or both MyoD and Ki-67 (0.4±0.4 vs 1.3±0.7 cells/mm², P=0.29) in non-ischemic non-treated gastrocnemius muscle from both E-sel/AAV and LacZ/AAV groups (0.8±0.5 vs 1.3±0.7 cells/mm², P=0.78). These findings indicate that at baseline, normal skeletal muscle has minimal regenerative activity. With tissue ischemia, however, skeletal muscle progenitor cells are activated and proliferate. This response can be significantly potentiated by E-sel/AAV gene therapy.

E-Sel/AAV is Associated with Increased Myh7+ Myofiber Differentiation

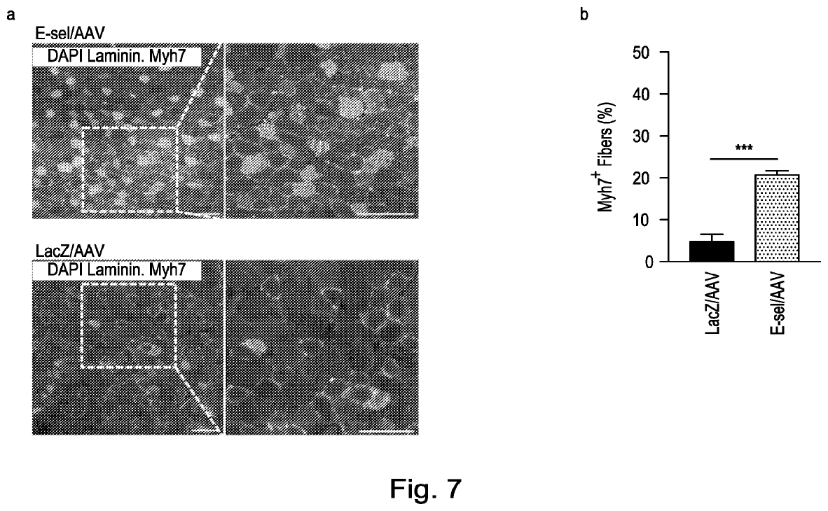

This study assessed whether treatment with E-sel/AAV influenced the relative distribution of myofiber type in regenerating muscle. Specifically, immunofluorescence staining for myosin heavy peptide 7 (Myh7), the myosin heavy chain isomer expressed in type I or slow-twitch myofibers was performed (FIG. 7A). Under normal conditions, Myh7 is not expressed in mouse gastrocnemius. In ischemic gastrocnemius, however, treatment with E-sel/AAV gene therapy can increase the proportion of Myh7⁺ fibers compared to LacZ/AAV control vector (21.0±0.7% vs 4.9±1.5%, P<0.001) (FIG. 7B).

### Induced Membrane-Bound E-Selectin Upregulation in Human Skeletal Myocytes

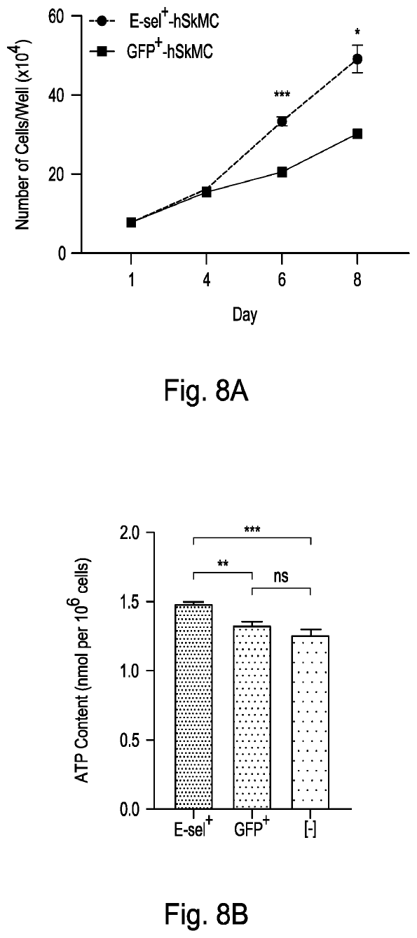

Human skeletal muscle cells (hSkMC) were transduced with E-selectin viral vector gene transfer or GFP gene transfer as a control. Following transduction, 5×10⁴ E-selectin+-hSkMC and GFP+-hSkMC were seeded in 6-well plates and proliferation was assessed by cell counting. Transgene expression was confirmed by flow cytometry and ATP content was measured after lysis of 1×10⁶ cells. Cell counting showed enhanced proliferation of E-selectin+-hSkMC (18.3±3.3×10⁴ cells) by Day 7 compared to GFP+-hSkMC controls (11.8±1.5×10⁴ cells) (p=0.02). There was also significantly greater ATP content in E-selectin+-hSkMC (1.48±0.03 nmol) compared to GFP+-hSkMC (1.33±0.04 nmol, p=0.003) and untransduced hSkMC (1.26±0.04 nmol, p<0.001). As shown in FIGS. 8A and 8B, in vitro overexpression of E-selectin in hSkMC increases both cellular proliferation (FIG. 8A) and ATP content (FIG. 8B).

### Induced E-Selectin Upregulation in Human Mesenchymal Stem Cells

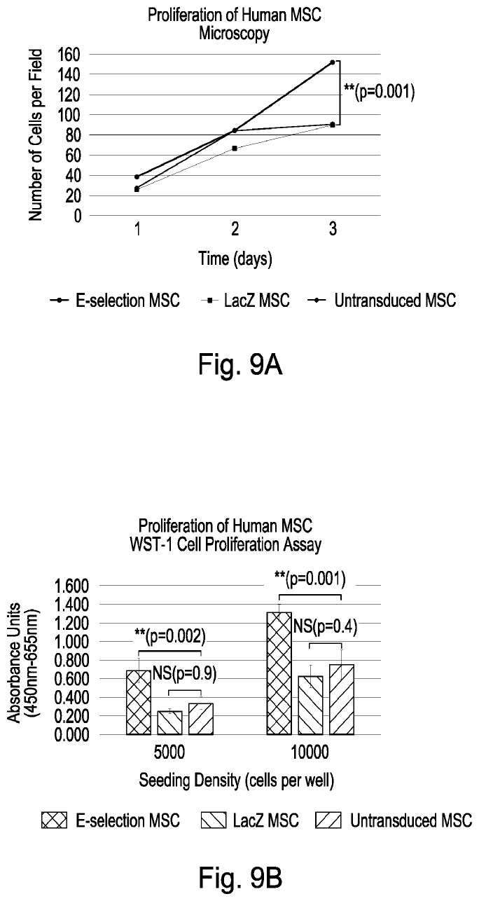

Human adipose tissue-derived mesenchymal stem cells (hMSC) were transduced with E-selectin/lentivirus or LacZ/lentivirus as control. Successful Following transduction, 2×104 E-selectin+-MSC and LacZ+-MSC were seeded in 6-well plates. Cell counting showed enhanced proliferation of E-selectin+-MSC by Day 3 compared to GFP+-MSC control. There was also significantly greater ATP content in E-selectin+-MSC compared to LacZ+-MSC and untransduced MSC. Overall, in vitro virally induced overexpression of E-selectin in human MSC promotes cellular proliferation as measured by microscopy and WST-1 assay. See FIG. 9.

### Discussion

Peripheral artery disease (PAD) is the manifestation of systemic atherosclerosis in the extremities. PAD affects 8.5 million people in the United States and more than 200 million worldwide [1]. Symptomatic patients with PAD typically present with exertional calf pain known as intermittent claudication [1]-[3]. The pathophysiology of intermittent claudication in PAD is related to both impaired perfusion and skeletal muscle dysfunction [4], [5]. Histologically, decreased size and abnormal morphology of myofibers correlates with functional impairments such as calf muscle strength and walking distance [6]. Neovascularization depends on recruitment of endothelial progenitor cells (EPCs), remodeling of the tissue microenvironment, and coordination of endothelial sprouting [7]. On the other hand, skeletal muscle regeneration requires activation, proliferation, and differentiation of muscle stem cells known as satellite cells. Both local tissue cells and progenitor cells recruited from circulation, arriving from distant niches such as the bone marrow are required to participate in order to achieve net muscle gain. The over-expression of cell-surface, membrane-bound E-Selectin, using a viral vector gene delivery method, leads to direct cell activation, independent of neovascularity and with normal oxygen and nutrient supplementation (FIGS. 8 and 9). Nevertheless, there is considerable overlap between these two processes during regeneration of ischemic muscle.

Several growth factors have been shown to play a role in regulation of both neovascularization and skeletal muscle regeneration. Vascular endothelial growth factor (VEGF) secreted in response to tissue hypoxia and primarily drives endothelial tip cell migration via Notch signaling and increased expression of Notch ligands such as delta-like 4 (DLL4) [8], [9]. However, there is also VEGF-mediated crosstalk between the vascular and muscle stem cell niche. Satellite cell-derived VEGF regulates the proximity of blood vessels to satellite cells while endothelial cells maintain satellite cell self-renewal [10]. VEGF also promotes fusion of myogenic cells into myotubes and protects against apoptosis [11]. Similarly, fibroblast growth factor (FGF) and hepatocyte growth factor (HGF) act synergistically as mitogens for activated satellite cells, and HGF specifically stimulates early entry of satellite cells into the cell cycle [12]. The angiopoietin family of growth factors, and specifically Ang-1, has also been implicated in both remodeling of blood vessels and myoblast differentiation [13], [14]. In practice, however, clinic trials of FGF, HGF, and VEGF have shown modest benefit for improving limb perfusion and wound healing in patients with PAD and CLTI [15]-[20]. However, the data provided herein demonstrated muscle regeneration without introduction of a soluble factor; and achieved direct cell activation of human skeletal muscle cells and Mesenchymal stem Cells, both needed for musculoskeletal healing and regeneration. Rather vector gene transfer of the membrane-bound E-Selectin adhesion molecule, was used to activate multiple local and recruited circulating cell types that contribute to net muscle regeneration. The combined in vivo and in vitro data show that both direct and indirect cell-cell mechanisms are at play and that circulation/angiogenesis is not required (though is also beneficially improved by E-Selectin gene therapy) for direct skeletal muscle cell activation and direct activation of Mesenchymal Stem Cells (as shown in FIGS. 8 and 9).

In one aspect of this study, an AAV vector was used to therapeutically increase E-selectin expression in ischemic mouse hindlimb muscle. First, the efficacy of AAV vector for high-level in vivo transgene expression was confirmed. Next, the experiment demonstrated the benefit of E-sel/AAV gene therapy for improving ischemic hindlimb reperfusion and functional recovery. In prior work, a FVB mice was used to create a hindlimb gangrene model and showed that E-sel/AAV can help restore blood flow and reduce severity of tissue loss [24]. In contrast, the C57BL/6 mice used in this study are more resistant than FVB and even more so than BALB/c strains [27], [28]. As such, C57BL/6 mice do not develop toe or foot necrosis after femoral artery coagulation. Thus, allowing to test hindlimb grip strength as a novel endpoint in addition to treadmill exercise capacity.

Upon observing a benefit of E-sel/AAV on functional recovery of skeletal muscle, the effect of E-selectin overexpression on myofiber regeneration was examined. Adult skeletal muscle regeneration is driven by activation of muscle stem cells known as satellite cells. Satellite cells reside between the muscle sarcolemma and basal lamina and characteristically express the transcription factor Pax7 [29], [30, p. 5]. Muscle injury activates the normally quiescent satellite cells to enter the cell cycle and then divide asymmetrically generating a progeny of committed precursor myoblasts while maintaining a self-renewing pool of satellite cells [31]-[33]. The differentiation of satellite cells into myoblasts and myofibers is coordinated by myogenic regulatory factors (MRFs) which comprise a family of basic helix-loop-helix transcription factors including MyoD, Myf5, myogenin, and MRF4 [34]. Sequential expression of MyoD and Myf5 coincides with satellite cell activation and proliferation and is required for myotube fusion and expression of myosin heavy chain (MyHC) [35], [36]. Thus, the finding that E-sel/AAV increased the number of cells expressing both MyoD and the proliferation marker Ki-67 suggests that this gene therapy may enhance myogenesis by increasing proliferation of muscle progenitor cells.

In addition to enhancing proliferation of skeletal muscle cell precursors, E-selectin overexpression was determined to be associated with an increased proportion of Myh7⁺ myofibers. The Myh7 gene codes for MyHC-β/slow and is preferentially expressed in type I or slow-twitch oxidative fibers in the heart and skeletal muscle [47]. Type I fibers are adapted for endurance and aerobic metabolism and have greater mitochondrial and myoglobin content than fast-twitch glycolytic-oxidative (IIA, Myh2) and glycolytic (IIB/IIX, Myh4/Myh1) fibers. Whereas the distribution of fiber type varies across species and muscle group, fiber-type switching can be induced to a varying extent by activity and metabolic changes. In mice, the calcineurin-nuclear factor of activated T cells (NFAT) signaling cascade has been implicated in activity-dependent fast-to-slow fiber-type switching via increased expression of myoglobin [48] and enzymes responsible for mitochondrial oxidative phosphorylation and lipid metabolism [49], [50]. In human patients, chronic ischemia leads to preferential denervation and oxidative damage to type II fibers and instead leads to muscle weakness and exercise impairment [52], [53]. In contrast, it was observed that treatment with E-sel/AAV in a mouse hindlimb ischemia model increased the proportion of type I fibers but also improved limb perfusion and function. Without wishing to be bound by specific theories, this discrepancy may be explained by differences in muscle fiber distribution in human gastrocnemius which contains a mix of type I and type II fibers compared to mouse gastrocnemius which predominantly consists of type II fibers.

The mechanisms by which E-selectin overexpression affects skeletal muscle regeneration remain to be elucidated. E-selectin is primarily involved in inflammatory and thrombotic processes via regulation of rolling and extravasation of circulating neutrophils and monocytes. E-selectin signaling is also key for trafficking of bone marrow-derived EPCs to areas of ischemia and wound healing for vasculogenesis. With regards to skeletal muscle, exercise can induce expression of endothelial cell adhesion molecules (CAMs) such as intercellular CAM 1 (ICAM-1), vascular CAM 1 (VCAM-1), and E-selectin in human skeletal muscle [38]. E-selectin has been shown to induce mitogen-activated protein kinase (MAPK) signaling in cultured endothelial cells [37]. In synectin-deficient and atherosclerotic mice, restoration of ERK1/2 activation via suppression of phosphoinositide 3-phosphate (PI3K) signaling has also been shown to stimulate arteriogenesis [39]. Regarding fiber-type switching, E-selectin mediated downstream activation of MEK1 and extracellular signal-related kinase 1/2 (ERK1/2) was also been shown to induce type 1 slow-twitch phenotype and protects against muscle damage in a mouse dystrophy model [51].

However, AAV does not readily transduce quiescent satellite cells [40]. Whereas ischemia may render satellite cells more receptive to transduction with AAV, this has not been demonstrated previously. Without wishing to be bound by specific theories, the observed effects of E-sel/AAV on activation of myogenic precursors is likely due to paracrine signaling from other resident or recruited cells. Alternatively, it is the consequence of improved tissue angiogenesis. Recently, it was shown that E-sel/AAV gene therapy can modulate the angiogenic and inflammatory gene expression profile of ischemic muscle. Most notably, E-sel/AAV upregulated a number of angiogenic factors including interleukin 6 (IL-6), tumor necrosis factor α (TNF-α), and monocyte chemoattractant protein 1 (MCP-1) [24]. These same factors are also expressed by satellite cells in response to muscle injury [41]. IL-6 is an essential regulator of skeletal muscle hypertrophy in response to muscle lengthening [42], [43] and promotes satellite cell proliferation via autocrine and paracrine signaling via janus kinase (JAK)/signal transducer and activator of transcription (STAT). Macrophage chemoattractant protein 1 (MCP-1, Ccl2), on the other hand, mediates the recruitment of monocytes to ischemic tissue [44]. Inflammatory (M1) macrophages are the dominant cell population up to 21 days after ischemic insult and play a key role in both collateral vessel formation and skeletal muscle regeneration. Therapeutic administration of M1 macrophages can increase myofiber size, decrease fibrosis, and increase contractile force in ischemic muscle [45]. While this study did not assess inflammation, no difference in infiltration of Mac-2⁺ macrophages [23] or CD3⁺ T cells [24] was previously observed in muscle treated with E-sel/AAV compared to LacZ/AAV. Other cell types such as mesenchymal stem cells (MSCs) have been shown to interact with satellite cells which can induce MSC myogenic commitment [46].

This study demonstrates that E-sel/AAV gene therapy can modulate the physiological response to ischemia. Additional studies seek to identify the precise mechanisms of E-selectin-mediated activation of muscle progenitor cells and fiber-type switching, as well as assess the in vitro effects of E-selectin overexpression on skeletal muscle cell proliferation, gene expression, and metabolism.

### CONCLUSIONS

This study confirms the efficacy of intramuscular E-sel/AAV gene therapy for therapeutic angiogenesis in a mouse hindlimb ischemia model. In addition to improving muscle perfusion, E-sel/AAV enhances proliferation of myogenic precursors and is associated with increased proportion of type I/slow-twitch myofibers in regenerating ischemic muscle. Altogether, these effects correlate with improved exercise capacity and suggest a potential role for E-sel/AAV gene therapy as a nonsurgical adjunct for patients with life-limiting PAD.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims. Further, those skilled in the art will recognize, or be able to ascertain, using no more than routine experimentation, numerous equivalents to the specific embodiments described specifically herein. Such equivalents are intended to be encompassed in the scope of the following claims.

### REFERENCES

[1] Polonsky et al., JAMA—J. Am. Med. Assoc., vol. 325, no. 21, pp. 2188-2198, 2021.
[2] Hamburg et al., Circ. J., vol. 81, no. 3, pp. 281-289, 2017.
[3] Bauersachs et al., Cardiovasc. Ther., vol. 2019, pp. 1-9, 2019.
[4] McDermott, et al., J. Am. Geriatr. Soc., vol. 55, no. 3, pp. 400-406, 2007.
[5] McDermott et al., Arterioscler. Thromb. Vasc. Biol., pp. 2577-2585, 2020.
[6] Koutakis, et al., J. Surg. Res., vol. 196, no. 1, pp. 172-179, 2015.
[7] Carmeliet et al., Nature, vol. 473, no. 7347, pp. 298-307, 2011.
[8] Apte, et al., Cell, vol. 176, no. 6, pp. 1-35, 2019.
[9] Liu, et al., Mol. Cell. Biol., vol. 23, no. 1, pp. 14-25, 2003.
[10] Verma, et al., Stem Cell, vol. 23, no. 4, pp. 530-543.e9, 2018.
[11] Arsic, et al., Mol. Ther., vol. 10, no. 5, pp. 844-854, 2004.
[12] Sheehan et al., J. Cell. Physiol., vol. 181, no. 3, pp. 499-506, 1999.
[13] McClung, et al., Front. Physiol., vol. 6, 2015.
[14] Youn et al., Sci. Rep., vol. 8, no. 1, p. 12323, 2018.
[15] Baumgartner et al., Circulation, vol. 97, no. 12, pp. 1114-1123, 1998.
[16] Isner et al., J. Vasc. Surg., vol. 28, no. 6, pp. 964-975, 1998.
[17] Comerota, et al., J. Vasc. Surg., vol. 35, no. 5, pp. 930-936, 2002.
[18] Kusumanto, et al., Hum. Gene Ther., vol. 17, no. 6, pp. 683-691, 2006.
[19] Morishita, et al., Curr. Gene Ther., vol. 20, no. 1, pp. 25-35, 2020.
[20] Germani, et al., Cardiovasc. Ther., vol. 27, no. 4, pp. 289-304, December 2009.
[21] Liu, et al., Sci. Rep., vol. 6, pp. 1-11, 2016.
[22] Parikh, et al., J. Surg. Res., vol. 228, pp. 68-76, 2018.
[23] Quiroz, et al., JVS Vasc. Sci., vol. 2, pp. 20-32, 2021.
[24] Ribieras, et al., Front. Cardiovasc. Med., vol. 9, p. 929466, 2022.
[25] Clément et al., Mol. Ther.—Methods Clin. Dev., vol. 3, p. 16002, 2016.
[26] Ribieras, et al., J. Vis. Exp., no. 181, p. e63284, 2022.
[27] Chalothorn et al., Physiol. Genomics, vol. 30, no. 2, pp. 179-191, 2007.
[28] Parikh, et al., Ann. Vasc. Surg., vol. 48, pp. 222-232, 2018.
[29] Seale, et al., Cell, vol. 102, pp. 770-786, 2000.
[30] Gunther, et al., Cell Stem Cell, vol. 13, no. 5, pp. 590-601, 2013.
[31] Péault, et al., Mol. Ther., vol. 15, no. 5, pp. 867-877, 2007.
[32] Shinin, et al., Nat. Cell Biol., vol. 8, no. 7, pp. 677-687, 2006.
[33] Kuang, et al., Cell, vol. 129, no. 5, pp. 999-1010, 2007.
[34] Cornelison et al., Dev. Biol., vol. 191, no. 2, pp. 270-283, 1997.
[35] Paoni, et al., Physiol. Genomics, vol. 11, no. 3, pp. 263-272, 2022.
[36] Yamamoto, et al., Stem Cell Rep., vol. 10, no. 3, pp. 956-969, 2018.
[37] Hu, et al., J. Immunol., vol. 165, no. 4, pp. 2142-2148, August 2000.
[38] Strömberg, et al., J. Appl. Physiol., vol. 122, no. 5, pp. 1145-1154, May 2017.
[39] Ren, et al., J. Clin. Invest., vol. 120, no. 4, pp. 1217-1228, April 2010.
[40] Arnett, et al., Mol. Ther.—Methods Clin. Dev., vol. 1, p. 14038, 2014.
[41] Hansen, et al., J. Am. Heart Assoc., vol. 10, no. 21, p. e022127, 2021.
[42] Toth, et al., PLoS ONE, vol. 6, no. 3, p. e17392, 2011.
[43] Serrano, et al., Cell Metab., vol. 7, no. 1, pp. 33-44, 2008.
[44] Shireman, et al., J. Surg. Res., vol. 134, no. 1, pp. 145-157, 2006.
[45] Hsieh, et al., J. Vasc. Surg., vol. 67, no. 6, pp. 1908-1920.e1, 2018.
[46] Kowalski, et al., Stem Cell Res. Ther., vol. 9, no. 1, p. 258, 2018.
[47] Schiaffino et al., Physiol. Rev., vol. 91, no. 4, pp. 1447-1531, 2011.
[48] Naya, et al., J. Biol. Chem., vol. 275, no. 7, pp. 4545-4548, 2000.
[49] Ryder, et al., J. Biol. Chem., vol. 278, no. 45, pp. 44298-44304, 2003.
[50] Long, et al., J. Biol. Chem., vol. 282, no. 3, pp. 1607-1614, 2007.
[51] Boyer, et al., JCI Insight, vol. 4, no. 10, p. e127356, May 2019.
[52] Koutakis, et al., Redox Biol., vol. 2, pp. 921-928, 2014.
[53] Regensteiner, et al., Circulation, no. 87, pp. 413-421, 1993.

What is claimed is:

1. A method of inducing myogenesis in non-ischemic atrophied muscle in a subject in need thereof, comprising administering to the subject a hybrid adeno-associated virus (AAV) comprising a nucleotide sequence encoding an E-selectin, in amount effective to induce myogenesis in the non-ischemic atrophied muscle of the subject.

2. The method of claim 1, wherein the subject is not suffering from critical limb ischemia.

3. The method of claim 1, wherein the subject does not have peripheral artery disease (PAD).

4. The method of any one of claims 1-3, wherein the hybrid AAV comprises AAV serotype 2 (AAV2) inverted terminal repeats (ITRs) and a capsid from an AAV other than serotype 2.

5. The method of claim 4, wherein the capsid is an AAV serotype 8 capsid and the hybrid AAV is an AAV2/8.

6. The method of claim 4, wherein the capsid is an AAV serotype 9 capsid and the hybrid AAV is an AAV2/9.

7. The method of any one of claims 1-6, wherein the AAV is administered intramuscularly to the atrophied muscle in the subject.

8. A method of treating a muscle tear in non-ischemic muscle tissue in a subject in need thereof, comprising administering to the subject a hybrid adeno-associated virus (AAV) comprising a nucleotide sequence encoding an E-selectin, in amount effective to treat the muscle tear in the subject.

9. The method of claim 8, wherein the subject is not suffering from critical limb ischemia.

10. The method of claim 8, wherein the subject does not have peripheral artery disease (PAD).

11. The method of any one of claims 8-10, wherein the hybrid AAV comprises AAV serotype 2 (AAV2) inverted terminal repeats (ITRs) and a capsid from an AAV other than serotype 2.

12. The method of claim 11, wherein the capsid is an AAV serotype 8 capsid and the hybrid AAV is an AAV2/8.

13. The method of claim 11, wherein the capsid is an AAV serotype 9 capsid and the hybrid AAV is an AAV2/9.

14. The method of any one of claims 8-13, wherein the AAV is administered intramuscularly to the atrophied muscle in the subject.

15. A method of improving the outcome of a connective tissue reattachment procedure in a subject in need thereof comprising administering to the subject a hybrid adeno-associated virus (AAV) comprising a nucleotide sequence encoding an E-selectin in an amount effective to improve the outcome of procedure, wherein the subject is not suffering from critical limb ischemia.

16. The method of claim 15, wherein the procedure is rotator cuff repair, Achilles tendon repair, patellar-patella tendon repair, medial cruciate ligament (MCL) reconstruction, anterior cruciate ligament (ACL) reconstruction, ulnar collateral ligament (UCL), meniscus repair, or labrum repair.

17. The method of claim 15, wherein the connective tissue is a ligament, tendon, meniscus or a labrum.

18. The method of claim 15 or claim 17, wherein the connective tissue is a tendon.

19. The method of any one of claims 15-18, wherein the hybrid AAV comprises AAV serotype 2 (AAV2) inverted terminal repeats (ITRs) and a capsid from an AAV other than serotype 2.

20. The method of claim 19, wherein the capsid is an AAV serotype 8 capsid and the hybrid AAV is an AAV2/8.

21. The method of claim 19, wherein the capsid is an AAV serotype 9 capsid and the hybrid AAV is an AAV2/9.

22. The method of any one of claims 15-21, wherein the AAV is administered intramuscularly to the atrophied muscle in the subject.
